(19) United States
(12) Patent Application Publication
(10) Pub. No.: US 2022/0183720 A1
(43) Pub. Date: Jun. 16, 2022

(54) METHOD AND DEVICE FOR REMOVING AN ECTOPARASITE FROM THE SKIN

(71) Applicant: Ákos Elemér GOMBOS, Leányfalu (HU)

(72) Inventor: Ákos Elemér GOMBOS, Leányfalu (HU)

(21) Appl. No.: 17/605,039

(22) PCT Filed: Jan. 31, 2020
(86) PCT No.: PCT/HU2020/050005

§ 371 Date: Oct. 20, 2021

(30) Foreign Application Priority Data
May 13, 2019 (HU) P1900151
Aug. 13, 2019 (HU) P1900286
Dec. 11, 2019 (HU) P1900418

(51) Int. Cl. A61B 17/50; A61B 50/30

(52) U.S. Cl. CPC A61B 17/50; B29C 2035/0827; A61B 2017/505; A61B 50/30

(57) ABSTRACT

The invention relates to a method for removing ectoparasites from the skin. During the method a plastic state medium which is able to solidify when it is exposed to electromagnetic radiation is applied around the ectoparasite at least partially penetrated the skin. Then the plastic state medium is exposed to electromagnetic 5 radiation and solidified. The ectoparasite is removed from the skin together with the solidified cover. The invention further relates to a device for removing ectoparasites from the skin. The device comprises a container (5) containing liquid or gel state medium (4) which is able to solidify when it is exposed to electromagnetic radiation (12), a radiating means (6) 10 applicable to emit electromagnetic radiation (12) and an energy source (7).

The present invention relates to a method and device for removing ectoparasites from the skin.

Solutions for tick removal in which the tick is grasped at its head or body by means of a tweezers-like device are known.

Hungarian patent application HU219024 describes an apparatus for removing a tick from the skin. The device has a cylindrical grip and a calotte-like sole. The sole is formed as an embossed scoop. The thickness of the sole symmetrical and perpendicular to the elongated axis of the grip is at most measured so that when it is pushed between the mouth part and the body of the tick penetrated the skin it does not tear off the head part of the tick. In the middle of the sole a V-form cut is provided which ends in a widened, advantageously circular opening around the point of intersection of the axis of the grip.

Patent application P0600896 describes a removing device for ticks of small size, especially for larvae or nymphs. The device comprises a shank and a tip connected to the shank with or without a neck. The thin side of the tip forms a 0.4-0.6 mm long blunt edge perpendicular to the symmetry plane of the device. The blunt edge and at least all the edges on the tip are rounded off at least by a 0.2 mm radius.

Patent application WO2019022626 A1 describes a device for neutralizing ectoparasites. In order to prevent a release of toxic substances into a host body by an ectoparasite during its removal the device comprises a neutralizing freezing medium.

Patent application WO 9500077 A1 describes an ectoparasite remover and method for removing ectoparasites e.g. ticks from a host organism. The remover uses an anesthetizing gas injected into a receptacle to cause the ectoparasite to voluntarily withdraw from the host organism.

The proper use of the known tick and other ectoparasite removing devices requires very good eyesight and manual skills. Most of the solutions suggest to take hold of the body or the basis part of the tick for removal and tear the proboscis having dentitions off the skin of the host organism. Compared to the body of the tick relatively great pressing force is required to remove the tick from the skin so during use of the conventional devices the body of the tick may be smashed. In this manner the risk of transmitting the pathogens carried by the tick into the host body is greater. Consequently, it is important that use of the device should be safe even for an inexperienced person.

The aim of the invention is to provide a method and device for removing an ectoparasite as soon as it is discovered. Use of the method does not require special experience, skill.

Further, an important aim is that by means of the device used in the method removal of the ectoparasites discovered possibly through feeling on parts of the body which are difficult to reach or difficult to see should be performed efficiently. In this manner the method and the device can be used successfully by children and even by visually impaired persons.

It has been realized that if the tick and other ectoparasites are encircled by a soft, quick-binding material and locked to the surface of the skin, the ectoparasite can be removed easily after solidification of the material. There is no need for exerting mechanic or physical force on the ectoparasite which would damage it giving rise to a possible infection caused by its saliva. Thus, the tick and other ectoparasites are removed from the host body so that pressing force is not applied on the body of the ectoparasite.

The invention is a method for removing ectoparasites from the skin. In the method plastic—liquid or gel state—medium which is able to solidify when it is exposed to electromagnetic radiation is applied around the ectoparasite which at least partially penetrated the skin, then the plastic state medium is exposed to electromagnetic radiation and solidified and the ectoparasite is removed from the skin together with the solidified cover.

Further, the invention is a device for removing ectoparasites from the skin according to the method. The device comprises a container containing liquid or gel state medium which is able to solidify when it is exposed to electromagnetic radiation, a radiating means applicable to emit electromagnetic radiation and an energy source.

Some advantageous embodiments of the invention are described in the appended claims.

The method and the device according to the invention will be described with reference to the accompanying drawings in which:

FIG. 1 shows the side view of an ectoparasite, in this case a tick embedded in the skin, where the skin and the host body are shown in a partial sectional view;

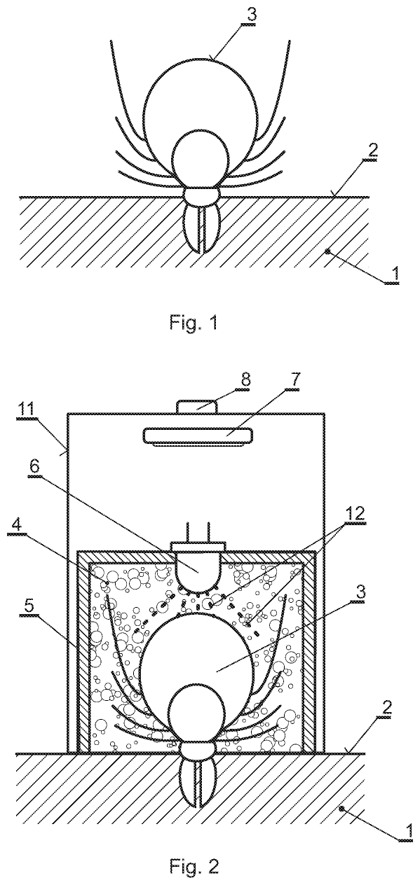

FIG. 2 is a partial sectional side view showing the first step of the process of removing the ectoparasite according to FIG. 1 using a container filled with the medium irradiated by electromagnetic radiation;

FIG. 3 is a partial sectional side view showing the first step of the process of removing the ectoparasite according to FIG. 1 using a container filled with the medium irradiated by electromagnetic radiation and also illuminated with a light-source;

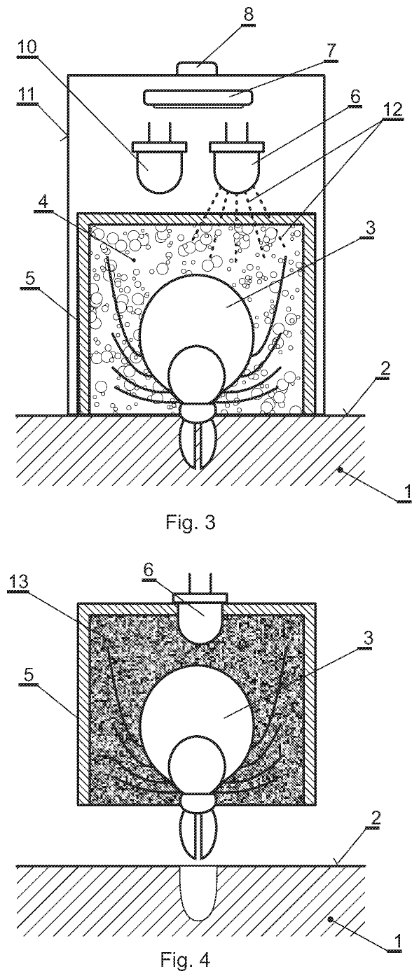

FIG. 4 is a partial sectional side view showing the removal of the ectoparasite according to FIG. 2 subsequent to the solidification of the medium;

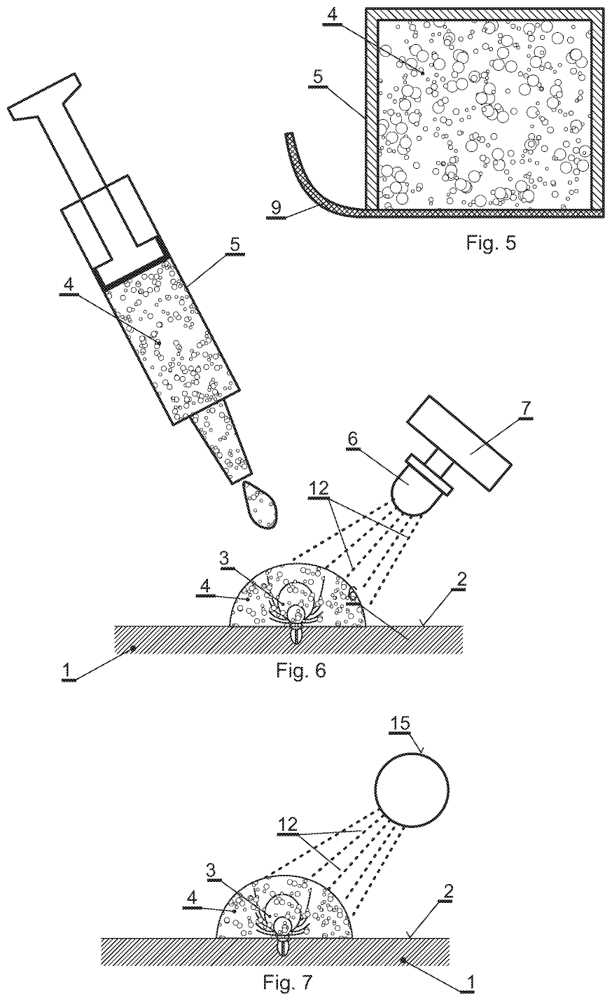

FIG. 5 shows the side sectional view of the container covered by a closing foil;

FIG. 6 is a partial sectional side view of a container realized as a syringe, here an independent light source is used for illumination;

FIG. 7 shows the partial sectional side view of the process of FIG. 6 wherein the Sun is used as the light source;

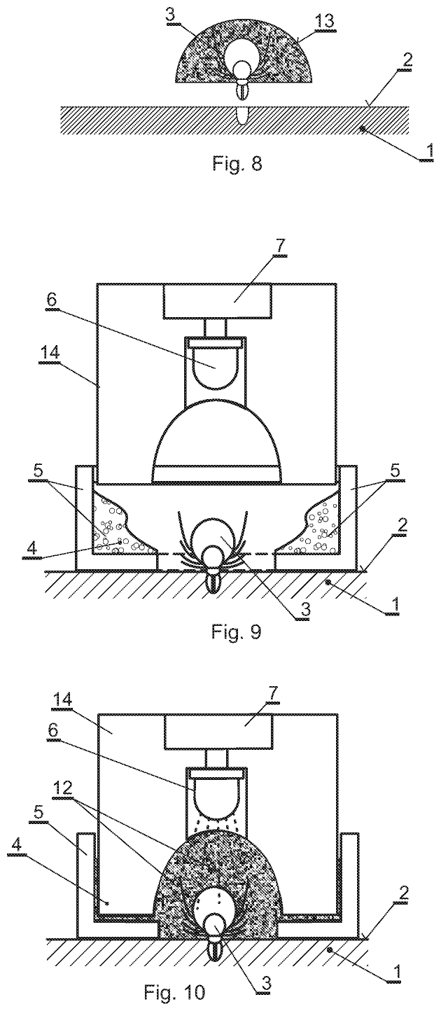

FIG. 8 is a partial sectional side view where the ectoparasite is removed subsequent to the process according to FIG. 6 or 7;

FIG. 9 shows the partial sectional side view of the container surrounding the ectoparasite and the housing having a rigid wall;

FIG. 10 shows the partial sectional side view of the container surrounding the ectoparasite and the housing having a rigid wall subsequent to pressing the medium out by means of the container and after solidification of the cover.

Operation of the device is explained through implementation of the method.

The device according to the present invention is applicable to remove ectoparasites 3 for example ticks (FIG. 1) from the skin 2. The device comprises a container 5 containing liquid or gel state medium 4 which is able to solidify when it is exposed to electromagnetic radiation 12, a radiating means 6 applicable to emit electromagnetic radiation 12 and an energy source 7 preferably provided with an activating switch 8. Advantageously, the device is provided with a subsidiary light source 10 for illuminating the ectoparasite 3 making its removal easier. Light source 10 emitting visible light is instrumental in proper positioning of the device when it is used (FIG. 3). In an advantageous embodiment the radiating means 6 emitting electromagnetic radiation may directly be in contact with medium 4 for solidification (FIG. 2). Advantageously, the component parts of the device may be arranged in a casing 11. The electromagnetic radiation 12 may be e.g. radio wave, microwave, infrared light, ultraviolet radiation or radiation of the Sun 15. It depends on which type of electromagnetic radiation 12 promotes the solidification of the medium 4 in the shortest time.

Container 5 is made of a material permeable for electromagnetic radiation 12. Container 5 containing medium 4 is sealed with a manually removable closing foil 9 which is naturally peeled off before use of the device (FIG. 5).

In an advantageous embodiment the device may be provided with means giving light or sound signals when solidification of the medium 4 is accomplished. In this case the energy source 7 is provided with a timer which emits a light or sound signal when the time for solidification of medium 4 terminates. This can be solved by coupling a LED diode and/or a buzzer. This is not shown in the Figures as solution of this is well within the knowledge of those skilled in the art. Advantageously the component parts of the device may be arranged in a casing 11.

If a gel state medium 4 is used which maintains the drop- or bubble-shape when it is applied on ectoparasite 3 then container 5 may have the form of a syringe. In this case the electromagnetic radiating means 6 and the energy source 7 may be fixed to the syringe-form container 5 (this solution is not shown as realization of it is clear). Naturally, the electromagnetic radiating means 6 and energy source 7 may be arranged independent of container 5 (FIGS. 6 and 7).

In a further embodiment the plastic state medium 4 is enclosed in a partially soft walled container 5 which can be fit and fixed—preferably by gluing—around the ectoparasite 3 while the light source 10 and the energy source 7 are arranged in a housing 14 having a rigid wall. The housing 14 fits on the soft wall of container 5.

During use of the device the ectoparasite 3 at least partially penetrated the skin 2 is encircled by the plastic either liquid or gel state medium 4 which is able to solidify when it is exposed to electromagnetic radiation 12. The plastic state medium 4 is placed into container 5 which is open at least one of its sides and the container 5 with its open side is placed on the ectoparasite 3 so that the plastic medium 4 encircles the portion of the ectoparasite 3 being on the surface of the skin 2 (FIGS. 2 and 3). Then the plastic state medium 4 is exposed to electromagnetic radiation 12 for solidification. The ectoparasite 3 fixed in the solidified medium 4 is removed from the skin 2 together with the solid cover 13 (FIG. 4).

According to another embodiment of the device the plastic state medium 4 is placed in a container 5 realized as a syringe. A drop of the medium 4 is placed on ectoparasite 3 so that the plastic state medium 4 encircles the portion of the ectoparasite 3 being on the surface of the skin 2 (FIGS. 6 and 7). Then the plastic state medium 4 is exposed to electromagnetic radiation 12 for solidification. The ectoparasite 3 fixed in the solidified medium 4 is removed from the skin 2 together with the solid cover 13 (FIG. 8).

In a further embodiment of the device the plastic state medium 4 is placed into container 5 which fits around ectoparasite 3 and is fixed preferably by means of glue. Light source 10 and energy source 7 are arranged in the housing 14 having a rigid wall (FIG. 9) and the medium 4 is pressed out by means of the housing 14 so that the plastic medium 4 encircles the portion of the ectoparasite 3 being on the surface of the skin 2 (FIG. 10). Then the plastic state medium 4 is exposed to electromagnetic radiation 12 for solidification. The ectoparasite 3 is removed from the skin 2 together with the solid cover 13.

The basis of the method according to the invention is that liquid or gel state medium 4 is used which when exposed to electromagnetic radiation 12 radio wave, microwave, infrared light, ultraviolet radiation or radiation of the Sun solidifies. The medium 4 in its plastic state forms a thick coating on the surface of the body of the ectoparasite 3 e.g. tick then after solidification of the medium 4 induced by an external factor a solid cover 13 is produced around the body of the ectoparasite 3.

The body of the ectoparasite 3 can be removed together with the solidified medium 4 without the body of the ectoparasite being crushed so it cannot inject its bodily fluid in the host body.

The device according to the invention ensures that the medium 4 (e.g. synthetic resin) which solidifies when exposed to electromagnetic radiation 12 can reach directly the ectoparasite 3. Also, it ensures the solidification of medium 4 when it is irradiated e.g. with ultraviolet light. According to one embodiment the medium 4 during its solidification also attaches together with the ectoparasite 3 to the container 5 of the device. In this manner when the container 5 of the device is removed from the host body, the ectoparasite 3 is also removed as it joins to the solidified cover 13.

In an advantageous embodiment of the device according to the invention only the medium 4 together with the container 5 is placed on the host body in such a manner that container 5 with its open side is placed on the ectoparasite 3 so that ectoparasite 3 immerses in medium 4.

Ectoparasite 3 may be stored together with the solidified medium 4 for subsequent e.g. health examination.

As it can be seen in FIG. 1 the ectoparasite 3 here the tick with its chelicera deeply penetrates in the host body 1. Typically, the head part is positioned under the surface of the skin 2 of the host body 1 while its body is positioned above the surface of the skin 2 of the host body 1. According to an advantageous embodiment of the device shown in FIG. 2 the radiating means 6 emitting electromagnetic radiation 12 (e.g. ultraviolet light) is in contact directly with medium 4 which is able to solidify when exposed to electromagnetic radiation. In FIG. 3 another advantageous embodiment of the device usable in the method can be seen. Container 5 is placed on the skin 2 so that it encircles the ectoparasite 3. The ectoparasite is surrounded by the liquid or gel state medium 4 stored in container 5. The medium 4 solidifies when exposed to electromagnetic radiation 12 (e.g. ultraviolet light). Within the casing 11 of the device a light source 10 visible for human eyes is placed which promotes proper positioning of the device on ectoparasite 3 even in poor visibility. After positioning the electromagnetic radiation 12 (e.g. ultraviolet light) which is able to permeate through the wall of container 5 solidifies the medium 4 in a few seconds. The energy needed for the radiating means 6 is ensured by an external energy source 7. The energy source 7 may be an inherent part of the device and can be activate by means of a switch 8. After solidification of the medium 4 the container 5 together with the ectoparasite 3 can be removed from the surface of the skin 2 as it is shown in FIG. 4. After solidification of the medium 4 a solid cover is formed around the ectoparasite 3 by means of which the container 5 and the ectoparasite 3 can be removed simultaneously from the host body 1 without deforming the body of the ectoparasite 3.

The solution according to FIGS. 6, 7 and 8 is different from the previously described ones only in that instead of placing the container 5 filled with the medium 4 on the ectoparasite, only medium 4 is applied on ectoparasite 3. After solidification of medium 4 the ectoparasite is removed together with the medium 4. The container 5 is represented by a syringe. The remaining medium 4 can be used again. This is advantageous as number of ectoparasite 3 may be removed by using a single container 5.

The housing 14 of the embodiments shown in FIGS. 9 and 10 surrounding the ectoparasite 3 has a spheroid form. Four sides of the container 5 may be closed and the lower part of it may be provided with a circular opening.

The advantage of the invention is that a method and device is provided with which an ectoparasite e.g. a tick embedded in the skin can be removed easily, without any risk. With the method very tiny (smaller than 1 mm) and larger (even 5 mm big) ectoparasites can be removed depending on the extent of embedding. Use of the device according to the invention does not require sharp eyes and precise movements not even in case of tiny ticks. Use of the device does not require training or experience, anyone can use it successfully even at the first occasion. With the device the tick can be removed within half a minute after the tick is noticed. As its size is small it can be a regular equipment of people living in tick infested areas or for those taking trips.

Further advantages of the invention—as compared to the known solutions where tweezers are used—are that pressing force is not exerted on the body of the ectoparasite e.g. tick in this manner its saliva cannot get into the host body thus the infection is prevented. There is no need for exact placing of the device on the ectoparasite, it is enough if the ectoparasite gets into the space of the container having a diameter 2-10 times larger than that of the ectoparasite or it is surrounded by the gel. Due to this, children, visually impaired or old people can use it successfully. Further, due to its subsidiary light source the device can be used in dark, poor visibility conditions. It can be used quickly; a tick may be removed in 30 seconds after it is detected. The device may have the size of a penlight battery, so it can be carried easily.

From the descriptions of the advantageous embodiments it can be seen that the method according to the invention is applicable to remove an ectoparasite especially a tick safely for an unexperienced user without being skillful with his hand and even in poor visibility conditions.

1. Method for removing ectoparasites from the skin characterized in that during the method plastic—liquid or gel state—medium which is able to solidify when it is exposed to electromagnetic radiation is applied around the ectoparasite at least partially penetrated the skin, then the plastic state medium is exposed to electromagnetic radiation and solidified and the ectoparasite is removed from the skin together with the solidified cover.

2. Method according to claim 1 characterized in that the plastic state medium is placed into a container which is open at least one of its sides and the container with its open side is placed on the ectoparasite so that the plastic medium encircles the portion of the ectoparasite being on the surface of the skin then the plastic state medium is exposed to electromagnetic radiation until it solidifies and the ectoparasite is removed from the skin together with the solid cover.

3. Method according to claim 1 characterized in that the plastic state medium is placed in a container realized as a syringe, then the medium is applied on the ectoparasite so that the plastic medium encircles the portion of the ectoparasite being on the surface of the skin then the plastic state medium is exposed to electromagnetic radiation until it solidifies and the ectoparasite is removed from the skin together with the solid cover.

4. Method according to claim 1 characterized in that the plastic state medium is placed into a container which is fixed preferably by gluing around the ectoparasite, a light source and an energy source are arranged in a housing having a rigid wall and the medium is pressed out by means of the housing so that the plastic medium encircles the portion of the ectoparasite being on the surface of the skin, then the plastic state medium is exposed to electromagnetic radiation until it solidifies and the ectoparasite is removed from the skin together with the solid cover.

5. Device for removing ectoparasites from the skin for implementing the method according to claim 1 characterized in that the device comprises a container (5) containing liquid or gel state medium (4) which is able to solidify when it is exposed to electromagnetic radiation (12), a radiating means (6) applicable to emit electromagnetic radiation (12) and advantageously it further comprises an energy source (7).

6. Device according to claim 5 characterized in that said container (5) is made of a material permeable for electromagnetic radiation (12).

7. Device according to claim 5 or 6 characterized in that said container (5) containing medium (4) is sealed with a manually removable closing foil (9).

8. Device according to any of claims 5-7 characterized in that said device is provided with means giving light or sound signals when solidification of the medium (4) is accomplished.

9. Device according to any of claims 5-8 characterized in that said device is provided with a subsidiary light source (1)0 for lighting the ectoparasite (3).

10. Device according to claim 5 characterized in that said container (5) is realized as a syringe and said electromagnetic radiating means (6) and said energy source (7) are fixed to said container (5).

11. Device according to claim 5 characterized in that said container (5) is realized as a syringe and said electromagnetic radiating means (6) and said energy source (7) are arranged independent of said container (5).

12. Device according to claim 5 characterized in that said plastic state medium (4) is enclosed in a partially soft walled container (5) which can be fit and fixed—preferably by gluing—around the ectoparasite (3) while said light source (10) and said energy source (7) are arranged in a housing (14) having a rigid wall, and said housing (14) fits on the soft wall of said container (5).

13. Device according to claim 5 characterized in that said radiating means is the Sun (15).
